(19) United States
(12) Patent Application Publication
(10) Pub. No.: US 2025/0000750 A1
(43) Pub. Date: Jan. 2, 2025

(54) COMPOSITIONS, GARMENTS COMPRISING SAME AND METHODS FOR MOISTURISING SKIN

(71) Applicant: QILTA LIMITED, Dublin (IE)

(72) Inventors: Manuj Sharma, Dublin (IE); Niall Murphy, Dublin (IE); James Dunne, Dublin (IE)

(21) Appl. No.: 18/692,294

(22) PCT Filed: Sep. 15, 2022
(86) PCT No.: PCT/EP2022/075672

§ 371 Date: Mar. 14, 2024

(30) Foreign Application Priority Data
Sep. 16, 2021 (GB) 2113271.7

(51) Int. Cl. A61K 8/02; A41D 27/00; A61K 8/06; A61K 8/31; A61K 8/34; A61K 8/37; A61K 8/46; A61Q 19/00

(52) U.S. Cl. CPC A61K 8/0204; A41D 27/00; A61K 8/062; A61K 8/31; A61K 8/34; A61K 8/345; A61K 8/37; A61K 8/46; A61Q 19/007

(57) ABSTRACT

An oil-in-water emulsion moisturising composition is described, a garment comprising said oil-in-water emulsion composition, and a method for application of said composition is described.

### FIELD

The present invention relates to moisturizer compositions, garments comprising same, and methods for moisturising the skin of a subject.

### BRIEF DESCRIPTION OF RELATED TECHNOLOGY

Sporting activities in general are undertaken in dedicated sporting apparel, including various garments that are exclusively designed for that sport, either for reasons of ergonomics, performance, comfort or a combination of the above.

It is not however uncommon for athletes, in particular those performing at the highest levels of their sport, to seek to further improve their performance by improving their garments in order to eke out performance gains and/or increased comfort or reduced fatigue, in order to allow the athlete to perform at an even higher level and/or for longer periods of sustained activity. In addition an athlete will look to find means to achieve a faster or more effective recovery following a sporting activity, which can allow the athlete to return to training sooner, or to train more intensely following the sporting activity.

One such example is long distance or endurance runners who, in addition to dedicated footwear, will also often have high performance socks which perform various functions such as wicking moisture away from the skin, regulating temperature and/or having anti-microbial or other performance enhancing materials incorporated into the sock or other garment, for example a calf sleeve or the like. Even with all of the above many long-distance runners will implement additional treatments such as moisturising the skin on their feet in order to avoid blisters or other conditions, and directly following an activity, to improve recovery. This may be achieved by applying a moisturising composition, or any other medicinal topical composition, directly to the skin prior to applying a garment such as a sock or sleeve (e.g. a calf sleeve), or the moisturiser or other composition may be applied to the inner surface of the garment which has the benefit of allowing the user to prepare the garment in advance of commencing the sporting activity, and then to simply put on the garment and thereby apply the moisturiser or other composition to the area. The garment can then be applied to provide recovery and or relief to the feet or other body part of the athlete following their sporting activity. Application of the composition either directly onto the feet or initially onto the garment can however be a relatively messy procedure. In addition in such application techniques it can be difficult to apply the correct quantity of the moisturiser and to evenly distribute it on the inner surface of the garment.

US patent application publication number US20100175167 discloses a sock formed of a water proof material, which can be used to apply and maintain foot treatment products in contact with a wearer's foot.

U.S. Pat. No. 5,682,617 discloses a non-porous lightweight non-permeable stretchable elastic latex stocking for use in treating skin irritations of the leg by the application of treatment lotions to the affected skin area of the leg, said stocking serving to hold said lotion in contact with the irritated areas of said leg due to its elasticity, said elastic latex stocking having a thickness of from 0.003 to 0.005 inches and a modulus of elasticity of from 450 to 500. The stockings described therein are employed to provide covering for medicaments such as creams and pastes that may be prescribed for various conditions, and serve to keep dirt and other foreign matter away from the area being treated.

US patent application publication number US2006/0130216 discloses a garment such as a sock comprising skin engaging means having an inner surface arranged to engage in use a wearer's skin, and hydration means on the skin engaging means to hydrate the skin. The hydration means may be a moisturizing cream or lotion. The hydration means may be coated onto the inner surface of the skin engaging means.

U.S. Pat. No. 4,186,445 discloses a disposable cosmetic glove which comprises an outer glove shell of lotion impervious material and an inner glove lining of lotion absorbent material. The inner glove lining is preferably uniformly impregnated with a cosmetic lotion, however, the glove may be provided with a lining which is not impregnated thereby permitting a lotion of personal choice to be injected into the lining prior to use. In a preferred embodiment, the disposable glove comprises an outer shell of thermoplastic resin material to which a thin layer of polyether or polyester is bonded.

International patent application publication number WO2019224319 discloses a garment treatment system and a garment treatment method, and in particular a system and method for applying a coating of a fluid composition such as a moisturiser or the like to the inner surface of a garment such as a sock, calf sleeve, glove or the like in order to improve the functionality and/or performance of the garment, the system comprising a main body, a support provided on or formed integrally with the main body and about which at least an opening of the garment may be secured, and a fluid supply circuit operable to deliver a quantity of a fluid composition onto at least a portion of an interior surface of the garment when secured to the support.

Cosmetic compositions which have a cooling effect are known in the art. For example, international patent application publication number WO2006037448 describes a cosmetic composition having a long-lasting cooling effect for about 8 hours. The composition comprises a cooling complex consisting of 0.05-0.15% menthyl lactate, 0.1-0.2% menthyl PCA and 0.05-0.2% ethyl menthane carboxamide and a remainder up to 100% of further cosmetic auxiliaries, active agents or mixtures thereof, wherein all percentages are in % by weight relative to the composition's total weight.

The incorporation of anti-inflammatory agents in cosmetic compositions is also known. Components such as glycyrrhetinic acid, palmitoyl-ethanolamine, telmesteine, Vitis vinifera, ceramide-dominant barrier repair lipids and filaggrin breakdown products have considerable anti-inflammation properties through various mechanism, such as blocking cyclooxygenase activity and down regulating cytokines as well as proinflammatory prostanoid production, providing soothing effect on inflamed skin, such as in dermatitis.

US patent application publication no. 2008286390 discloses a skin care composition comprising moisturizers and a plurality of natural oils containing essential fatty acids, the natural oils being selected from the group consisting of borage oil, omega-3 oil, emu oil, evening primrose oil, safflower oil, hemp seed oil, flaxseed oil, and rosehip oil. The moisturizers include olive oil and glycerin. In addition, the skin care composition contains an anti-inflammatory agent, preferably aloe vera juice and aloe extract, and emulsifiers, stabilizers, emollients for softening the skin, and water. The skin care composition may also contain a fragrance and a preservative. Optionally, the skin care composition may contain menthol crystals.

A plethora of moisturisers are commercially available, with many indicating efficacy in the treatment of particular skin conditions, and/or problem areas. Scholl foot cream for example, is marketed as being effective for hydrating skin and providing softer and smoother skin. Scholl foot cream includes for example urea, vitamin E and provitamin B5 complex.

Biofreeze gel is a composition comprising menthol which acts as a cooling agent, and the gel is marketed as providing fast acting, long lasting pain relief for sore muscles, backaches sore joints and arthritis. Biofreeze spray is a similar composition which comprises 10 wt % menthol and is sold in a pressurised aerosol canister.

It would be desirable to provide a moisturiser which treats and prevents dryness, increases skin flexibility, provides a cooling effect, and an anti-inflammatory effect, thereby reducing aches and pains during sporting activity and in recovery thereafter.

### SUMMARY

In one aspect, the present invention provides an oil-in-water emulsion composition comprising:
an emollient which is present in an amount of from 1 wt % to 9 wt % based on the total weight of the composition;
a humectant comprising glycerine which is present in an amount of from 2% to 20 wt % based on the total weight of the composition;
a cooling agent selected from the group consisting of menthol, menthyl lactate, menthyl acetate, levomenthol, one or more menthol esters, and methyldiisopropyl propionamide; wherein said cooling agent is present in an amount of from 0.1 wt % to 5 wt %, optionally 0.1 wt % to 3 wt % based on the total weight of the composition;
an anti-inflammatory agent comprising dimethyl sulfone which is present in an amount of from 0.2 wt % to 10 wt % based on the total weight of the composition;
an emulsifier which is present in an amount of from 5 wt % to 10 wt %;
and water which is present in an amount of from 60 wt % to 75 wt % based on the total weight of the composition.

Suitably, the emollient is present in an amount of from 1 wt % to 5 wt % based on the total weight of the composition, suitably from 1.5 wt % to 4.5 wt %, preferably from 1.5 wt % to 3.5 wt % based on the total weight of the composition.

The emollient may comprise at least one of the following group of emollients: mineral oil, petrolatum, paraffin, ozokerite, microcrystalline wax, polyethylene, squalene and perhydrosqualene. Suitably, the emollient may comprise two of said group. For example, the emollient may comprise a combination of at least two of mineral oil, petrolatum and paraffin. Preferably, the emollient comprises petrolatum and paraffin.

The emulsifier may be present in an amount of from 5 wt % to 10 wt %, such as from 6 wt % to 9 wt %.

Suitably, the emulsifier is selected from one or more of:
(i) polyethylene glycol ethers of the general formula R—O—(—CH₂—CH₂—O—)n-R′, where R and R′, independently of one another, are branched or unbranched alkyl or alkenyl radicals and n is a number from 10 to 80,
(ii) polyglyceryl fatty acid esters, and
(iii) an acrylate-C₁₀₋₃₀ acrylate crosspolymer.

The emulsifier may comprise a polyethylene glycol ether of alcohols selected from isocetyl alcohol (e.g., Isoceteth-20), isostearyl alcohol (e.g., Isosteareth-20), cetyl alcohol (e.g., Ceteth-20), oleyl alcohol (e.g., Oleth-20) and cetearyl alcohol (e.g., Ceteareth-20).

Suitably, the emulsifier comprises at least one polyethylene glycol ether and at least one polyglyceryl fatty acid ester. The emulsifier may optionally further comprise an acrylate-C₁₀₋₃₀ acrylate crosspolymer.

The oil-in-water emulsion composition may further comprise a preservative. Suitably, the preservative may be present in an amount of from about 0.01 wt % to 5 wt %, preferably from 0.1% to 2 wt %, such as from 0.5 wt % to 1.5 wt % based on the total weight of the composition.

The preservative may be selected from the group consisting of: a parahydroxybenzoic acid, methylparaben, propylparaben, benzyl alcohol, phenoxyethanol, ethylhexylglycerin, octanediol, hexanediol, pentandiol, sorbitan caprylate, capralyl glycol, caprylhydroxamic acid, phenoxyethanol triethylene glycol, sodium benzoate, and 2-bromo-2-nitro-1,3-propanediol (bronopol). The preservative may comprise at least one of said group, or combinations thereof. Preferably the preservative comprises phenoxyethanol.

The oil-in-water emulsion composition may comprise a pH modifier. The composition may have a pH in the range of from 5.0 to 7.0, preferably 5.3 to 6.3. Advantageously,

Suitably, the glycerine is present in an amount of from 10 wt % to 16 wt % based on the total weight of the composition, preferably from 12 wt % to 14 wt % based on the total weight of the composition.

The cooling agent may be present in an amount of from 0.5 to 1.5 wt % based on the total weight of the composition, preferably from 0.8 to 1.2 wt % based on the total weight of the composition.

The dimethyl sulfone may be present in an amount of from 2 to 8 wt % based on the total weight of the composition, preferably from 3 to 7 wt % such as from 4 to 7 wt % based on the total weight of the composition.

The water may be present in an amount of from 63 to 73 wt % based on the total weight

of the composition, preferably from 65 to 70 wt % based on the total weight of the composition.

The composition may have a viscosity in the range of from 500 mPa·s to 35000 mPa·s wherein the composition has a viscosity in the range of from 500 mPa·s to 35000 mPa·s when measured at 20° C. using a Brookfield viscometer (Model DV-I+) with a type 93 spindle and a rotation speed of 30 rpm, preferably wherein the viscosity is in the range of from 500 mPa·s to 3,000 mPa·s when measured at 20° C.

The composition may optionally further comprise one or more additives in an amount of from 0.01 wt % to 10 wt %, suitably in an amount of from 0.1 wt % to 10 wt %, such as from 0.5 wt % to 8 wt %, for example from 1 wt % to 6 wt %.

In another aspect the present invention provides a method for moisturising the skin of a subject comprising the following steps: spraying an oil-in-water composition as described herein onto a garment, and applying said garment to the skin of said subject, such that the oil-in-water composition applied to said garment wets the skin of said subject. Suitably, the method involves application of 8 to 12 mL of a composition as described herein to an inner skin facing surface of said garment, for example from 9 to 11 mL, preferably 10 ml of said composition.

Suitably, the garment is a sock, sleeve (e.g. a calf sleeve) or glove. Suitably, the garment comprises an inner layer and an outer layer, and the composition of the invention is at least partially coated on an inner (skin facing) surface of said inner layer.

In yet a further aspect, the present invention provides a garment comprising a composition as described herein. Suitably, the garment comprises an inner layer and an outer layer. The inner layer is arranged to engage a wearer's skin when in use, said an inner surface of said inner layer is at least partially coated with a composition as described herein.

Suitably, the inner layer comprises a polyamide material, preferably nylon 66 (poly[imino(1,6-dioxohexamethylene) iminohexamethylene]). The inner layer may further comprise up to 10 wt % elastane based on the total weight of the inner layer, suitably from 0.5 wt % to 10 wt % elastane based on the total weight of the inner layer, for example from 1 wt % to 5 wt %.

The inner layer may be formed from knitted yarn. The knitted yarn may include 2-ply yarn with a linear density in the range of from 50 dtex to 100 dtex. The knitted yarn may include 2-ply yarn wherein a single ply of said yarn comprises from 50 to 90 filaments, preferably from 60 to 80 filaments, such as from 65 to 75 filaments. Suitably each ply of said 2-ply yarn comprises from 50 to 90 filaments, preferably from 60 to 80 filaments, such as from 65 to 75 filaments.

The inner layer may comprise nylon 66 and elastane. The inner layer may comprise nylon 66 in an amount of from 80 wt % to 99 wt %, such as from 90 to 99 wt % based on the total weight of the inner layer. The inner layer may comprise elastane in an amount of 0.5 wt % to 10 wt %, such as 1 wt % to 5 wt % based on the total weight of the inner layer.

The inner layer may be formed from knitted yarn wherein the yarn comprises 2-ply nylon 66 yarn and 2-ply elastane yarn.

The 2-ply nylon 66 yarn may have a linear density in the range of from 50 dtex to 100 dtex, preferably from 75 dtex to 85 dtex, such as from 75 to 80 dtex. 1 dtex is 1 gram per 10,000 metres.

The 2-ply elastane yarn may have a linear density in the range of from 50 dtex to 100 dtex, preferably from 75 dtex to 85 dtex, such as from 75 to 80 dtex.

Suitably a single ply of the 2-ply nylon 66 yarn comprises from 50 to 90 filaments, preferably from 60 to 80 filaments, such as from 65 to 75 filaments, for example 68 filaments.

Suitably a single ply of the 2-ply elastane yarn comprises from 15 to 35 filaments, preferably from 20 to 30 filaments, such as from 20 to 25 filaments, for example 23 filaments.

In still a further aspect the present invention provides a garment as described above, comprising the composition of the invention. The garment comprises an inner layer and an outer layer, wherein a composition as described herein is partially coated on an inner surface of said inner layer, said inner surface being a skin facing surface when said garment is worn by a user.

The garment may for example be a sleeve (e.g. a calf sleeve), a glove or a sock.

### DETAILED DESCRIPTION

The present invention provides a moisturiser which treats and prevents skin dryness, increases skin flexibility, provides a cooling effect, and an anti-inflammatory effect, thereby reducing aches and pains during sporting activity and in recovery thereafter.

The composition of the invention comprises three active components in particular:
(i) a humectant comprising glycerine, which facilitates moisturising of the skin, thereby treating and preventing dryness and increasing skin flexibility;
(ii) a cooling agent selected from the group consisting of menthol, menthyl lactate, menthyl acetate, levomenthol, one or more menthol esters and methyldiisopropyl propionamide, which provides a cooling effect and provides pain relief for sore muscles; and
(iii) an anti-inflammatory component comprising dimethyl sulfone, which reduces inflammation and provides pain relief.

The first active, glycerine is a humectant that can be used to treat and prevent dry, rough, scaly, itchy skin and minor skin irritations (e.g. callouses, hard skin, minor cuts/scrapes). The compositions of the invention facilitate absorbing and retaining of moisture in the skin, thereby softening the skin when used regularly. This increases the flexibility in the skin, making it less prone to problems that amateur and professional athletes may commonly suffer from e.g. cuts, scrapes, and blisters.

Cooling agents such as menthol have been used as analgesics in topical preparations for many years. As a topical agent, the majority of the effects of cooling agents such as menthol are acute and immediate, imparting a cooling effect, initially stimulating pain receptors and then desensitizing them to provide relief. It is believed such cooling agents, and menthol in particular, have a capacity to act centrally on pain pathways. Menthol is also known to have antimicrobial activity and deodorizing properties.

The third active component i.e. the anti-inflammatory component comprises methylsulfonylmethane (MSM), also known as dimethyl sulfone and methyl sulfone. It is an organic sulfur-containing compound that occurs naturally in a variety of fruits, vegetables, grains, and animals. Studies have shown it has anti-inflammatory benefits, acting through multiple pathways decreasing proliferation of several pro-inflammatory cytokines. Studies undertaken have largely involved oral use and have been small in size, but improvements in joint pain, stiffness, and swelling have all been reported with consistent usage. Unlike the instant effect elicited by the cooling agent component, which provides immediate benefit on contact, the impact of MSM is more gradual, long-lasting, aiding recovery and helping to reduce the likelihood of injury.

The inventive compositions described herein are unique for the combination of these three ingredients. Collectively in the composition of the invention, there is provided an oil-in-water emulsion which is effective at reducing aches/pain both immediately after sports but also helping reduce the likelihood of injury and aiding recovery after exertion for example through endurance/performance exercise/activities such as running over a longer period with consistent usage.

Though each of the active components is known per se, the combination of said components to provide an effective moisturising formulation which is sprayable, physically stable, stable to microbiological burden, and which solves the above-identified problems required considerable optimisation. The oil-in-water emulsion composition of the invention has been formulated through extensive trials and testing to exhibit rheological properties, viscosity and consistency that allow for its optimal application to a garment such as sock, sleeve, glove or similar garment, while maintaining stability of the formulation and eliciting the desired moisturizing, cooling and pain-relieving effects.

As outlined above, the present invention provides an oil-in-water emulsion composition comprising:
an emollient which is present in an amount of from 1 wt % to 9 wt % based on the total weight of the composition;
a humectant comprising glycerine which is present in an amount of from 2% to 20 wt % based on the total weight of the composition;
a cooling agent selected from the group consisting of menthol, menthyl lactate, menthyl acetate, levomenthol, one or more menthol esters, and methyldiisopropyl propionamide; wherein said cooling agent is present in an amount of from 0.1 wt % to 5 wt %, optionally 0.1 wt % to 3 wt % based on the total weight of the composition;
an anti-inflammatory agent comprising dimethyl sulfone which is present in an amount of from 0.2 wt % to 10 wt % based on the total weight of the composition;
an emulsifier which is present in an amount of from 5 wt % to 10 wt %;
and water which is present in an amount of from 60 wt % to 75 wt % based on the total weight of the composition.

The oil-in-water emulsion composition may consist essentially of, or consist of:
an emollient which is present in an amount of from 1 wt % to 9 wt % based on the total weight of the composition;
a humectant comprising glycerine which is present in an amount of from 2% to 20 wt % based on the total weight of the composition;
a cooling agent selected from the group consisting of menthol, menthyl lactate, menthyl acetate, levomenthol, one or more menthol esters, and methyldiisopropyl propionamide; wherein said cooling agent is present in an amount of from 0.1 wt % to 5 wt %, optionally 0.1 wt % to 3 wt % based on the total weight of the composition;
an anti-inflammatory agent comprising dimethyl sulfone which is present in an amount of from 0.2 wt % to 10 wt % based on the total weight of the composition;
an emulsifier which is present in an amount of from 5 wt % to 10 wt %;
and water which is present in an amount of from 60 wt % to 75 wt % based on the total weight of the composition; and optionally one or more preservatives.

The composition of the invention is designed to moisturise, cool and reduce aches and pains when applied to skin, particularly when applied using a garment comprising an inner layer and an outer layer, wherein the composition of the invention is at least partially coated on an inner (skin facing) surface of the inner layer.

The moisturising efficacy of the composition is optimal when the amount of glycerine is present in an amount of from about 10 wt % to 16 wt %, preferably from 12 wt % to 14 wt % based on the total weight of the composition.

The cooling efficacy of the composition is optimal when the amount of cooling agent is present in an amount of from 0.5 wt % to 1.5 wt % based on the total weight of the composition, preferably from 0.8 wt % to 1.2 wt % based on the total weight of the composition.

In a preferred embodiment, the cooling agent comprises menthol and is present in 0.5 to 1.5 wt %, preferably 0.8 to 1.2 wt % based on the total weight of the composition.

The ability of the composition of the invention to reduce aches and pains is optimal when the anti-inflammatory agent comprising dimethyl sulfone is present in an amount of 2 to 8 wt %, preferably from 4 to 7 wt % based on the total weight of the composition.

In a particularly preferred embodiment, the oil-in-water composition of the invention comprises glycerine in an amount of from 10 wt % to 16 wt % based on the total weight of the composition, the cooling agent comprises menthol and is present in an amount of from 0.5 wt % to 1.5 wt % based on the total weight of the composition and the anti-inflammatory agent comprising dimethyl sulfone is present in an amount of from 2 wt % to 8 wt % based on the total weight of the composition.

In a preferred embodiment the emollient comprises petrolatum and paraffin, in an amount of from 1 wt % to 9 wt %, based on the total weight of the composition.

If the amount of emollient is less than 1 wt % or greater than 9 wt % the rheological properties of the composition are negatively impacted, which can deleteriously impact how the composition behaves when applied to a garment, for example, leakage through a twin skin garment can increase.

The emulsifier is present in an amount of from 5 wt % to 10 wt % based on the total weight of the composition.

Examples of emulsifiers include polypropoxylated O/W emulsifiers where the polyethoxylated or polypropoxylated O/W coemulsifiers or emulsifiers is or are chosen from the group:
of fatty alcohol ethoxylates of the general formula R—O—(—CH₂—CH₂—O—)n-H where R is a branched or unbranched alkyl, aryl or alkenyl radical and n is a number from 10 to 50,
of ethoxylated wool wax alcohols,
of polyethylene glycol ethers of the general formula R—O—(—CH₂—CH₂—O—)n-R′, where R and R′, independently of one another, are branched or unbranched alkyl or alkenyl radicals and n is a number from 10 to 80,
of fatty acid ethoxylates of the general formula R—COO—(—CH₂—CH₂—O—)n-H, where R is a branched or unbranched alkyl or alkenyl radical and n is a number from 10 to 40,
of etherified fatty acid ethoxylates of the general formula R—COO—(—CH₂—CH₂—O—)n-R′, where R and R′, independently of one another, are branched or unbranched alkyl or alkenyl radicals and n is a number from 10 to 80,
of esterified fatty acid ethoxylates of the general formula R—COO—(—CH₂—CH₂—O—)n-C(O)—R′, where R and R′, independently of one another, are branched or unbranched alkyl or alkenyl radicals and n is a number from 10 to 80,
of polyethylene glycol glycerol fatty acid esters of saturated or unsaturated, branched or unbranched fatty acids and a degree of ethoxylation between 3 and 50,
of ethoxylated sorbitan esters with a degree of ethoxylation of from 3 to 100,
of cholesterol ethoxylates with a degree of ethoxylation between 3 and 50,
of ethoxylated triglycerides with a degree of ethoxylation between 3 and 150,
of alkyl ether carboxylic acids of the general formula R—O—(—CH₂—CH₂—O—)n-CH₂—COOH or cosmetically or pharmaceutically acceptable salts thereof, where R is a branched or unbranched alkyl or alkenyl radical having 5-30 carbon atoms and n is a number from 5 to 30,
of polyoxyethylene sorbitol fatty acid esters, based on branched or unbranched alkanalic or alkenalic acids and having a degree of ethoxylation of from 5 to 100, for example of the sorbeth type,
of alkyl ether sulfates or the acids on which these sulfates are based of the general formula R—O—(—CH₂—CH₂—O—)n-SO₃—H with cosmetically or pharmaceutically acceptable cations, where R is a branched or unbranched alkyl or alkenyl radical having 5-30 carbon atoms and n is a number from 1 to 50,
of fatty alcohol propoxylates of the general formula R—O—(—CH₂—CH(CH₃)—O—)n-H, where R is a branched or unbranched alkyl or alkenyl radical and n is a number from 10 to 80,
of polypropylene glycol ethers of the general formula R—O—(—CH₂—CH(CH₃)—O—)n-R′, where R and R′, independently of one another, are branched or unbranched alkyl or alkenyl radicals and n is a number from 10 to 80,
of propoxylated wool wax alcohols,
of etherified fatty acid propoxylates of the general formula R—COO—(—CH₂—CH(CH₃)—O—)n-R′, where R and R′, independently of one another, are branched or unbranched alkyl or alkenyl radicals and n is a number from 10 to 80,
of esterified fatty acid propoxylates of the general formula R—COO—(—CH₂—CH(CH₃)—O—)n-C(O)—R′, where R and R′, independently of one another, are branched or unbranched alkyl or alkenyl radicals and n is a number from 10 to 80,
of fatty acid propoxylates of the general formula R—COO—(—CH₂—CH(CH₃)—O—)n-H, where R is a branched or unbranched alkyl or alkenyl radical and n is a number from 10 to 80
of polypropylene glycol glycerol fatty acid esters of saturated or unsaturated, branched or unbranched fatty acids and a degree of propoxylation between 3 and 80,
of propoxylated sorbitan esters with a degree of propoxylation from 3 to 100,
of cholesterol propoxylates with a degree of propoxylation from 3 to 100,
of propoxylated triglycerides with a degree of propoxylation from 3 to 100,
of alkyl ether carboxylic acids of the general formula R—O—(—CH₂—CH(CH₃)—O—)n-CH₂—COOH or cosmetically or pharmaceutically acceptable salts thereof, where R is a branched or unbranched alkyl or alkenyl radical and n is a number from 3 to 50,
of alkyl ether sulfates or the acids on which these sulfates are based of the general formula R—O—(—CH₂—CH(CH₃)—O—)n-SO₃—H with cosmetically or pharmaceutically acceptable cations, where R is a branched or unbranched alkyl or alkenyl radical having 5-30 carbon atoms and n is a number from 1 to 50,
of fatty alcohol ethoxylates/propoxylates of the general formula R—O—Xn-Ym-H, where R is a branched or unbranched alkyl or alkenyl radical, where X and Y are not identical and in each case are either an oxyethylene group or an oxypropylene group, and n and m, independently of one another, are numbers from 5 to 50,
of polypropylene glycol ethers of the general formula R—O—Xn-Ym-R′, where R and R′, independently of one another, are branched or unbranched alkyl or alkenyl radicals, where X and Y are not identical and in each case are either an oxyethylene group or an oxypropylene group, and n and m, independently of one another, are numbers from 5 to 100,
of etherified fatty acid propoxylates of the general formula R—COO—Xn-Ym-R′, where R and R′, independently of one another, are branched or unbranched alkyl or alkenyl radicals, where X and Y are not identical and are in each case either an oxyethylene group or an oxypropylene group, and n and m, independently of one another, are numbers from 5 to 100, and
of fatty acid ethoxylates/propoxylates of the general formula R—COO—Xn-Ym-H, where R is a branched or unbranched alkyl or alkenyl radical, where X and Y are not identical and in each case are either an oxyethylene group or an oxypropylene group, and n and m, independently of one another, are numbers from 5 to 50.
of fatty alcohol ethoxylates of the general formula R—O—(—CH₂—CH₂—O—)n-H where R is a branched or unbranched alkyl, aryl or alkenyl radical and n is a number from 10 to 50,
of ethoxylated wool wax alcohols,
of polyethylene glycol ethers of the general formula R—O—(—CH₂—CH₂—O—)n-R′, where R and R′, independently of one another, are branched or unbranched alkyl or alkenyl radicals and n is a number from 10 to 80,
of fatty acid ethoxylates of the general formula R—COO—(—CH₂—CH₂—O—)n-H, where R is a branched or unbranched alkyl or alkenyl radical and n is a number from 10 to 40,
of etherified fatty acid ethoxylates of the general formula R—COO—(—CH₂—CH₂—O—)n-R′, where R and R′, independently of one another, are branched or unbranched alkyl or alkenyl radicals and n is a number from 10 to 80,
of esterified fatty acid ethoxylates of the general formula R—COO—(—CH₂—CH₂—O—)n-C(O)—R′, where R and R′, independently of one another, are branched or unbranched alkyl or alkenyl radicals and n is a number from 10 to 80,
of polyethylene glycol glycerol fatty acid esters of saturated or unsaturated, branched or unbranched fatty acids and a degree of ethoxylation between 3 and 50,
of ethoxylated sorbitan esters with a degree of ethoxylation of from 3 to 100,
of cholesterol ethoxylates with a degree of ethoxylation between 3 and 50,
of ethoxylated triglycerides with a degree of ethoxylation between 3 and 150,
of alkyl ether carboxylic acids of the general formula R—O—(—CH₂—CH₂—O—)n-CH₂—COOH or cosmetically or pharmaceutically acceptable salts thereof, where R is a branched or unbranched alkyl or alkenyl radical having 5-30 carbon atoms and n is a number from 5 to 30,
of polyoxyethylene sorbitol fatty acid esters, based on branched or unbranched alkanalic or alkenalic acids and having a degree of ethoxylation of from 5 to 100, for example of the sorbeth type,
of alkyl ether sulfates or the acids on which these sulfates are based of the general formula R—O—(—CH₂—CH₂—O—)n-SO₃—H with cosmetically or pharmaceutically acceptable cations, where R is a branched or unbranched alkyl or alkenyl radical having 5-30 carbon atoms and n is a number from 1 to 50,
of fatty alcohol propoxylates of the general formula R—O—(—CH₂—CH(CH₃)—O—)n-H, where R is a branched or unbranched alkyl or alkenyl radical and n is a number from 10 to 80,
of polypropylene glycol ethers of the general formula R—O—(—CH₂—CH(CH₃)—O—)n-R′, where R and R′, independently of one another, are branched or unbranched alkyl or alkenyl radicals and n is a number from 10 to 80,
of propoxylated wool wax alcohols,
of etherified fatty acid propoxylates of the general formula R—COO—(—CH₂—CH(CH₃)—O—)n-R′, where R and R′, independently of one another, are branched or unbranched alkyl or alkenyl radicals and n is a number from 10 to 80,
of esterified fatty acid propoxylates of the general formula R—COO—(—CH₂—CH(CH₃)—O—)n-C(O)—R′, where R and R′, independently of one another, are branched or unbranched alkyl or alkenyl radicals and n is a number from 10 to 80,
of fatty acid propoxylates of the general formula R—COO—(—CH₂—CH(CH₃)—O—)n-H, where R is a branched or unbranched alkyl or alkenyl radical and n is a number from 10 to 80,
of polypropylene glycol glycerol fatty acid esters of saturated or unsaturated, branched or unbranched fatty acids and a degree of propoxylation between 3 and 80,
of propoxylated sorbitan esters with a degree of propoxylation from 3 to 100,
of cholesterol propoxylates with a degree of propoxylation from 3 to 100,
of propoxylated triglycerides with a degree of propoxylation from 3 to 100,
of alkyl ether carboxylic acids of the general formula R—O—(—CH₂—CH(CH₃)—O—)n-CH₂—COOH or cosmetically or pharmaceutically acceptable salts thereof, where R is a branched or unbranched alkyl or alkenyl radical and n is a number from 3 to 50,
of alkyl ether sulfates or the acids on which these sulfates are based of the general formula R—O—(—CH₂—CH(CH₃)—O—)n-SO₃—H with cosmetically or pharmaceutically acceptable cations, where R is a branched or unbranched alkyl or alkenyl radical having 5-30 carbon atoms and n is a number from 1 to 50,
of fatty alcohol ethoxylates/propoxylates of the general formula R—O—Xn-Ym-H, where R is a branched or unbranched alkyl or alkenyl radical, where X and Y are not identical and in each case are either an oxyethylene group or an oxypropylene group, and n and m, independently of one another, are numbers from 5 to 50,
of polypropylene glycol ethers of the general formula R—O—Xn-Ym-R′, where R and R′, independently of one another, are branched or unbranched alkyl or alkenyl radicals, where X and Y are not identical and in each case are either an oxyethylene group or an oxypropylene group, and n and m, independently of one another, are numbers from 5 to 100,
of etherified fatty acid propoxylates of the general formula R—COO—Xn-Ym-R′, where R and R′, independently of one another, are branched or unbranched alkyl or alkenyl radicals, where X and Y are not identical and are in each case either an oxyethylene group or an oxypropylene group, and n and m, independently of one another, are numbers from 5 to 100, and
of fatty acid ethoxylates/propoxylates of the general formula R—COO—Xn-Ym-H, where R is a branched or unbranched alkyl or alkenyl radical, where X and Y are not identical and in each case are either an oxyethylene group or an oxypropylene group, and n and m, independently of one another, are numbers from 5 to 50.

Suitably, the emulsifier is selected from one or more of:
polyethylene glycol ethers of the general formula:
R—O—(—CH₂—CH₂—O—)n-R′, where R and R′, independently of one another, are branched or unbranched alkyl or alkenyl radicals and n is a number from 10 to 80,
polyglyceryl fatty acid esters, and an acrylate-C₁₀₋₃₀ acrylate crosspolymer.

In a particularly preferred embodiment the emulsifier comprises polyglyceryl-6 stearate and glyceryl stearate, and optionally an acrylate-C₁₀₋₃₀ acrylate crosspolymer. For example, the emulsifier may preferably comprise polyglyceryl-6-stearate, polyglyceryl-6 behenate, ceteareth-20, glyceryl stearate and optionally an acrylate-C₁₀₋₃₀ acrylate crosspolymer.

The acrylate-C₁₀₋₃₀ acrylate crosspolymer may have the following structure:

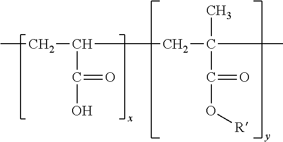

where R′ is an alkyl radical having 10 to 30 carbon atoms, and x and y are numbers which symbolize the respective stoichiometric content of each comonomer. Such copolymers comprise one or more monomers of acrylic acid, of methacrylic acid and one or more C₁₀₋₃₀ alkyl acrylates monomers. The INCI name of such compounds is “Acrylates/C₁₀₋₃₀ Alkyl Acrylate Crosspolymer”. Particularly advantageous are those available under the trade names Pemulen TR1, Pemulen TR2 and Pemulen EZ-4U polymeric emulsifier from Lubrizol.

As outlined above, the emulsifier is present in an amount of from 5 wt % to 10 wt %. When less than about 5 wt % of emulsifier is present the physicochemical stability of the composition was negatively impacted, and when greater than about 10 wt % of emulsifier was employed, leakage was observed when the composition was applied to the inner layer of a twin skin garment i.e. the composition leaked from the inner surface of the layer to the outer surface of the outer layer of the garment comprising an inner layer and an outer layer, in such tests 1.16 mL of the composition was applied to a 2500 mm² of a twin skin garment as outlined in greater detail below. Such leakage is undesirable as the amount of the composition in contact with the user will be reduced, and therefore efficacy negatively affected.

The oil-in-water composition of the invention comprises water in an amount of from 60wt % to 75 wt % based on the total weight of the composition. When less than about 60 wt % water is employed, the sprayability and rheological properties of the composition are negatively impacted, and when greater than about 75 wt % water is employed the physicochemical stability of the composition is negatively impacted. Compositions comprising greater than about 75 wt % tended to split (or layer) over time.

The composition of the invention may optionally further comprise one or more additives. The additives may be present in an amount of from 0.01 wt % to 10 wt % based on the total weight of the composition, suitably in an amount of from 0.1 wt % to 10 wt %, such as from 0.5 wt % to 8 wt %, for example from 1 wt % to 6 wt based on the total weight of the composition. Suitable additives will be known to those skilled in the art, though examples of suitable additives are provided below.

Suitably, the one or more additives is selected from the following:
(1) silicone oils, such as dimethyl polysiloxanes, methylphenyl polysiloxanes, water-soluble and alcohol-soluble silicone glycol copolymers;
(2) acetoglyceride esters, such as acetylated monoglycerides;
(3) ethoxylated glycerides, such as ethoxylated glyceryl monostearate;
(4) alkyl esters of fatty acids having 10 to 20 carbon atoms such as alkyl esters, which may include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate, including methyl, isopropyl, and butyl esters of fatty acids;
(5) alkenyl esters of fatty acids having 10 to 20 carbon atoms such as oleyl myristate, oleyl stearate, and oleyl oleate;
(6) fatty acids having 10 to 20 carbon atoms such as pelargonic, lauric, myristic, palmitic, stearic, isostearic, hydroxystearic, oleic, linoleic, ricinoleic, arachidic, behenic, and erucic acids;
(7) fatty alcohols having 10 to 20 carbon atoms such as lauryl, myristyl, cetyl, hexadecyl, stearyl, isostearyl, hydroxystearyl, oleyl, ricinoleyl, behenyl, erucyl alcohols, and 2-octyl dodecanol;
(8) fatty alcohols ethers including ethoxylated fatty alcohols of 10 to 20 carbon atoms such as the lauryl, cetyl, stearyl, isostearyl, oelyl, and cholesterol alcohols having attached thereto from 1 to 50 ethylene oxide groups or 1 to 50 propylene oxide groups;
(9) ether-esters such as fatty acid esters of ethoxylated fatty alcohols;
(10) lanolin and its derivatives such as lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases;
(11) polyhydric alcohols and polyether derivatives such as propylene glycol, dipropylene glycol, polypropylene glycols 2000 and 4000, polyoxyethylene polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly [ethylene oxide] homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C15-C18 vicinal glycol, and polyoxypropylene derivatives of trimethylolpropane;
(12) polyhydric alcohol esters including ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters;
(13) wax esters such as beeswax, spermaceti, myristyl myristate, and stearyl stearate;
(14) beeswax derivatives, such as polyoxyethylene sorbitol beeswax which are reaction products of beeswax with ethoxylated sorbitol of varying ethylene oxide content, forming a mixture of ether-esters;
(15) vegetable waxes including carnauba and candelilla waxes;
(16) natural or essential oils, including, vegetable oil, citrus oil, plant oil, fish oil, non-citrus fruit oil, nut oils, oils having flavours, perfume or scents; vegetable oils may include canola oil, corn oil, neem oil, olive oil, cottonseed oil, coconut oil, fractionated coconut oil, palm oil, nut oils, safflower oil, sesame oil, soybean oil, and sunflower oil; nut oils can be peanut oil, almond oil, cashew oil, hazelnut oil, macadamia oil, pecan oil, pine nut oil, pistachio oil, and walnut oil; citrus oils may include grapefruit seed oil, lemon oil, orange oil, sweet orange oil, tangerine oil, lime oil, mandarin oil, and the like; other natural or essential oils may include fish oil such as omega 3 oil, flaxseed oil (linseed oil), apricot oil, avocado oil, carrot oil, cocoa butter oil, coconut oil, fractionated coconut oil, hemp oil, papaya seed oil, rice bran oil, shea butter oil, tea tree seed oil, and wheat germ oil; additional natural or essential oils may include lavender oil, rosemary oil, tung oil, jojoba oil, poppy seed oil, shea butter, castor oil, mango oil, rose hip oil, tall oil; and natural or essential oils may include chamomile oil, cinnamon oil, citronella oil, eucalyptus oil, fennel seed oil, jasmine oil, juniper berry oil, raspberry seed oil, lavender oil, primrose oil, lemon grass oil, nutmeg oil, patchouli oil, peppermint oil, pine oil, rose oil, rose hip oil, rosemary oil, eucalyptus oil, tea tree oil, rosewood oil, sandalwood oil, sassafras oil, spearmint oil, and wintergreen oil;
(17) phospholipids, such as lecithin and derivatives;
(18) sterols such as cholesterol and cholesterol fatty acid esters; and
(19) amides such as fatty acid amides, ethoxylated fatty acid amides, and solid fatty acid alkanolamides. Examples of emollients that may be included in the compositions of the invention may include one or more of Ricinus communis (castor) seed oil, lanolin, aloe barbadensis leaf extract, Therbroma cacao (cocoa) seed butter, petrolatum, Euphorbia cerifera (candelilla) wax, honey, gerniol, camphor, cetyl esters, mineral oil, salicylic acid, Lavandula angustifolia (lavender) flower extract, phenol, Olea europaea (olive) oil, Camellia sinensis (green tea) seed oil, Prunus armeniacea (acai) fruit oil, Persea gratissima (avocado) oil, vegetable oil, palmitoyl isoleucine, Sambucus nigra (elderberry) fruit extract, Phoenix dactylifera (date) seed extract, Avandula stoechas (spanish lavender) extract, Spiraea ulmaria (meadowsweet) leave extract, Chamomilla recutita (chamomile) leaf extract, and Symphytum officinale (comfrey) leaf extract;
(20) solubilisers such as one or more of ethanol, propylene glycol, or polysorbates (e.g. those commercially available under the trade name Tween®).

In one preferred embodiment the oil-in-water emulsion composition comprises, consists essentially of, or consists of: petrolatum, paraffin, glycerine, menthol, dimethyl sulfone, polyglyceryl-6 stearate, polyglyceryl-6 behenate, glyceryl stearate, ceteareth-20, 2-phenoxyethanol, ethanol, citric acid and water.

Traditional moisturising compositions are applied directly to the skin, such as to the feet and limbs to extract their claimed benefits. In some circumstances such an application approach can be inconvenient. For example, the application of moisturiser products in cream, lotion, gel or spray form can create a greasy messy layer on the foot, and/or other limbs and hands from applying said products onto the area in question and every surface that comes in contact with the product also becomes coated with such a greasy messy layer. This is a particular issue with menthol-based products where inadvertent contact with sensitive body parts e.g. eyes can cause irritation.

This “messy nature” of the application process is especially inconvenient on site immediately before or after athletic events when athletes may be fatigued or have limited facilities available to them e.g. hand washing facilities. This is also the critical time for application i.e immediately after the athletic/sporting event, when such a product if applied is likely to be of most benefit.

When creams, gels and lotions are applied to feet and/or limbs, depending on particular area of application, a considerable amount of the applied product may get wasted through becoming absorbed by the individuals own clothing or other surfaces, before the product can reach and be absorbed by the skin reducing effectiveness and impact of the composition.

Finally, some people do not feel comfortable touching their feet for any reason, so the manual application of creams/gels/lotions would be considered an unpleasant experience for such people, who would prefer an alternative application methodology.

The compositions of the invention are preferably applied to a garment such as a sock, glove or sleeve (e.g. a calf sleeve) prior to application to the skin. The application of the moisturising compositions of the invention to the garment such as to a sock eliminates the messy process of manually applying a moisturiser to the feet onsite before and/or after an athletic event. The coated sock/sleeve can be pre-prepared for easy mess free application at home or in an instant on site at the start or finish of an event, with no inconvenience or waiting.

The compositions of the invention may preferably be applied by spraying using an applicator.

Suitable applicators are described in international patent application publication number WO2019224319, however, other applicators such as a spray pump applicator may also be used to apply the compositions of the invention, for example to a garment.

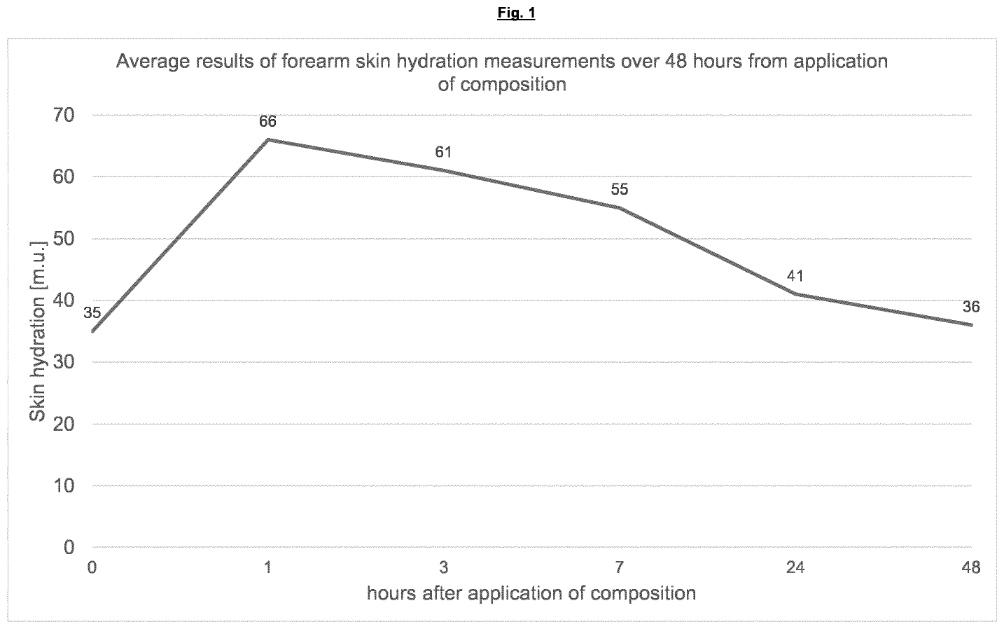

The applicator may for example be a garment treatment system as shown in FIGS. 1 and 2 of WO2019224319. The entire contents of WO2019224319 is hereby incorporated by reference, in particular the garment treatment system shown in FIGS. 1 to 11, and the description thereof on pages 5 to 11 of WO2019224319, and as claimed in claims 1 to 27 thereof.

In particular, the garment treatment system may comprise a main body; a support provided on or formed integrally with the main body and about which at least an opening of the garment may be secured; and a fluid supply circuit operable to deliver a quantity of a fluid composition onto at least a portion of an interior surface of the garment when secured to the support.

Preferably, the fluid supply circuit comprises a discrete reservoir of the fluid composition.

Preferably, the discrete reservoir comprises an aerosol canister.

Preferably, the discrete reservoir comprises a manually pressurisable canister.

Preferably, the fluid supply circuit comprises at least one nozzle operable to dispense a fluid directly or indirectly onto the interior surface of the garment.

Preferably, the at least one nozzle comprises an atomising nozzle.

Preferably, the at least one nozzle is operable to effect the unidirectional emission of the fluid composition.

Preferably, the at least one nozzle is operable to effect the multidirectional emission of the fluid composition.

Preferably, the at least one nozzle may be displaceable linearly and/or rotationally.

Preferably, the treatment system comprises a mast on which the at least one nozzle is mounted, the mast being retractably extendable relative to the main body.

Preferably, the mast is rotatable relative to the main body.

Preferably, the support comprises a frame on which the garment may be secured to retain the garment in an at least partially expanded state.

Preferably, the frame is rotatable relative to the main body.

Preferably, the treatment system comprises a housing defining the main body.

Preferably, the housing comprises a collar about an open end of the housing which defines the support.

Preferably, the collar is displaceable relative to the remainder of the housing to function as a switch.

Preferably, the treatment system comprises an air displacer operable to deliver air into an interior volume defined by the garment.

Preferably, the at least one nozzle is arranged such that the fluid dispensed from the nozzle is carried into the garment by the airflow generated by the air displacer.

Preferably, the air displacer is operable to generate an air flow rate of between r between 0.1 and 4000 l/m.

Preferably, the housing comprises a lower section containing the air displacer and a central section containing the fluid supply circuit.

Preferably, the upper and central sections are separated by an air permeable platform on which the fluid supply circuit is at least partly supported.

Preferably, the platform is displaceable between a first position and a second position wherein the air displacer is activated in response to the platform being displaced into the second position.

Preferably, the platform is biased towards the first position.

Preferably, the fluid supply circuit defines a mechanical connection between the support and the platform such as to facilitate displacement of the platform through displacement of the support.

Preferably, the fluid supply circuit comprises a fluid level indicator operable to illustrate the level of the fluid composition within the fluid supply circuit.

Preferably, the treatment system comprises a controller operable to effect actuation of the fluid supply circuit and/or air displacer in response to a user input.

Preferably, the controller comprises a timer operable to effect actuation of the fluid supply circuit and/or the air displacer for a set period of time.

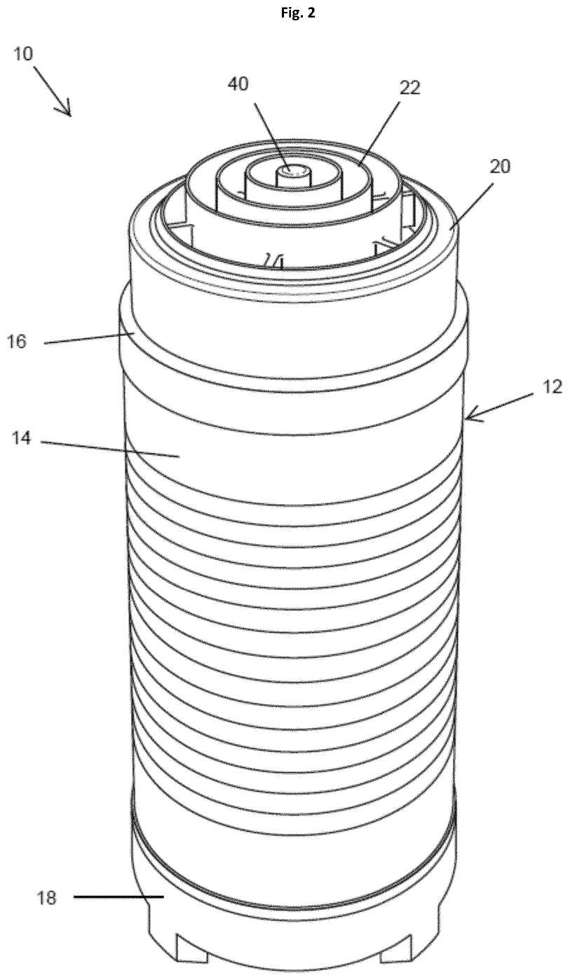

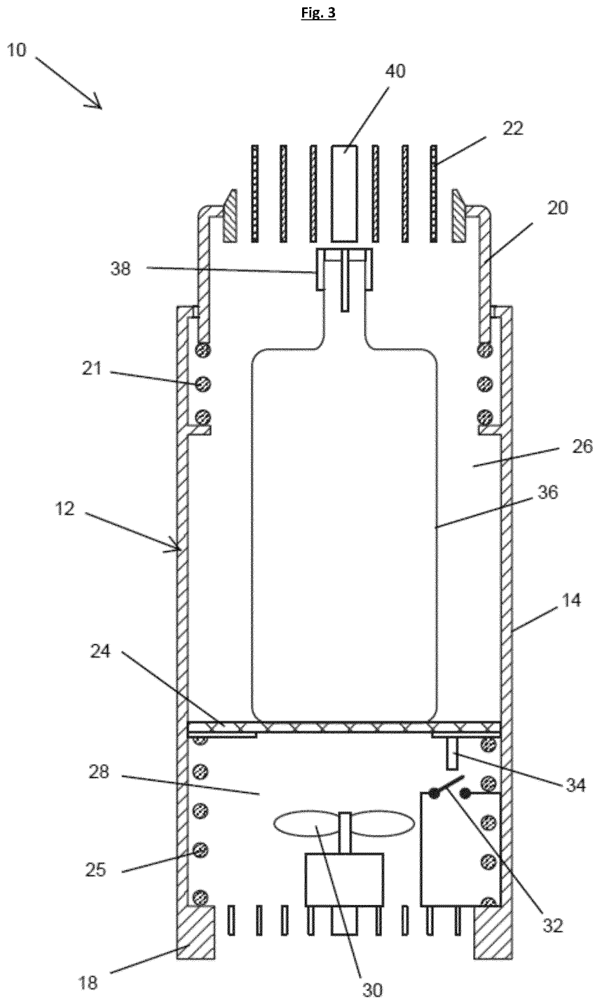

As outlined above, the applicator may be a garment treatment system as described in WO2019224319 (shown in FIGS. 1 and 2 thereof which are reproduced in FIGS. 2 and 3 respectively herein).

In FIG. 2 a garment treatment system as described in WO2019224319 is shown. The garment treatment system (10) comprises a main body defined by a housing (12) which in the embodiment illustrated is cylindrical in form and dimensioned to be portable in order to allow a user to transport and/or store the treatment system (10) when not in use, and to which housing (12) the sock(S) or other garment can be quickly and easily fitted to have a coating of the requisite fluid composition (i.e. the oil-in-water composition of the present invention) applied to an inner or skin contacting surface of the sock(S). It will of course be understood that the main body as defined by the housing (12) may be of any other shape, style and/or size.

The housing (12) comprises a cylindrical side wall (14) which in the embodiment illustrated is formed from a metal such as steel or aluminium, and may include ribbing, corrugations, fluting or the like in order to provide increased structural strength to the side wall (14), although such features are of course optional. It is also envisaged that the housing (12) may be formed from any other suitable material or combination of materials. The cylindrical housing (12) defines a first or upper end (16) which is open to an interior of the housing (12) and a second lower end or base (18) which is preferably also at least partially open to the interior of the housing (12) in order to allow air flow to be drawn into the housing (12) via the base (18) and expelled via the upper end (16) as will be described in detail hereinafter. It will however be appreciated from the following description of the configuration and operation of the system (10) that the housing (12) could be of any other suitable shape and/or dimension and various alternative openings (not shown) may be provided in order to facilitate the requisite flow of air as hereinafter described, or where such air flow through the housing (12) is not required, depending on the particular mechanism by which the fluid composition is to be applied to the interior surface of the garment as hereinafter described. In the first embodiment described and shown the housing (12) is approximately 200 mm in height and 75 mm in diameter, and is intended to be used in a substantially vertical orientation.

The housing (12) defines a support in the form of a collar (20) provided about the upper end (16) of the housing (12) which surrounds and/or retains a slotted or vended grill (22) forming a partial occlusion at the upper end (16) which permits air flow out of the housing (12) but prevents the ingress of physical objects into the interior of the housing (12). The collar (20) is mounted to the housing (12) in a manner which permits limited longitudinal or telescopic displacement of the collar (20) towards the base (18) against the action or bias of a first spring (21) located beneath the collar (20) in order to effect actuation of the system (10) as will be described in greater detail hereinafter.

Located on the interior of the housing (12) is an air permeable platform (24) which acts to divide the interior of the housing (12) into a central section or chamber (26) defined between the platform (24) and the grill (22), and a lower section or chamber (28) defined between the platform (24) and the base (18). The system (10) further comprises an air displacer, preferably in the form of a fan (30) located in the lower chamber (28), and preferably mounted to the base (18), although any other suitable arrangement may be employed.

A micro-switch (32) is also located in the lower chamber (28) and is operable to turn on and off the fan (30), an actuator (34) being located on the underside of the platform (24) in order to effect operation of the micro-switch (32). As with the collar (20), the platform (24) is longitudinally displaceable, against the action of a second spring (25) captured between the platform (24) and the base (18), which acts to return the platform (24) to its upper most position following a downward displacement thereof as hereinafter described. The second spring (25) has a lower spring rate than the first spring (21), so that the second spring (25) will compress prior to compression of the first spring (21). Suitable rails, tracks, slots or the like may be provided on the interior surface of the sidewall (14) and extending longitudinally, to guide and restrain the longitudinal displacement of the platform (24).

The system (10) additionally comprises a fluid supply circuit comprising a discreet reservoir (36) which in the embodiment illustrated is in the form of a sealed aerosol canister or a manually pressurisable reservoir or the like and which is supported on the platform (24) and is dimensioned such that a dispensing head (38) of the reservoir (36) is located directly beneath the grill (22), the dispensing head (38) comprising a nozzle (40) which projects upwardly through the grill (22). It will be understood that the nozzle (40) may project above or below the grill (22), depending on design and operation of the system (10). The reservoir (36) contains a discreet quantity of a liquid composition, for example a moisturiser or the like, under pressure and which may be dispensed from the reservoir (36) by a downward actuation of the dispensing head (38) which opens a valve (not shown) permitting an aerosolised dispensing of the liquid solution from the nozzle (40), preferably in discrete quantities, for example 10 ml and as is well known in the art. However it is also envisaged that alternative forms of the fluid supply circuit may be employed, for example comprising a pump and atomizing nozzle, to replace or augment the requirement for pressurisation of the discrete reservoir (36).

Alternatively, the applicator may be using a manual pump action mini trigger spray with pre-compression, for example a PZ Helios spray pump commercially available from Aptar®.

In a further aspect of the invention, the compositions described herein are applied to a garment, preferably a garment comprising an inner layer and an outer layer. The inner layer and outer layer are separate layers. The composition is applied to an inner surface of the inner layer.

Suitably, the composition of the invention is applied to the inner layer of a garment comprising an inner layer and an outer layer. In particular, the composition of the invention is applied to an inner surface of the inner layer of the garment, which the inner surface being a skin contacting surface when the garment is worn by a user.

Suitably, the garment comprises an inner layer comprising, consisting essentially of, or consisting of polyamide, such as nylon 66 (i.e. (poly[imino(1,6-dioxohexamethylene) iminohexamethylene]).

The inner layer may further comprise elastane. For example, the inner layer may be formed from polyamide and elastane.

Suitably, the inner layer comprises up to 10 wt % elastane, based on the total weight of the inner layer. For example, the inner layer may comprise 0.5 wt % to 10 wt % elastane, suitably 1 to 6 wt % elastane, or 1 wt % to 5 wt % based on the total weight of the inner layer.

The inner layer may be formed from a knitted material, such as knitted yarn. The inner layer may be formed from a knitted material comprising at least two different types of yarn, for example, yarn made from two different materials.

The inner layer may be formed from 2-ply multifilament yarn.

Suitably, the 2-ply multifilament yarn has a linear density in the range of from inner layer is formed from 50 dtex to 100 dtex, such as from 60 dtex to 90 dtex, preferably from 65 to 85 dtex, such as from 70 to 80 dtex. (1 dtex is 1 gram per 10,000 m of yarn).

As outlined above, the yarn is suitably 2-ply multifilament yarn. A strand or ply of yarn may for example comprise from 50 to 80 filaments, such as from 60 to 80 filaments, such as 65 to 75 filaments.

A preferred yarn is a 2-ply polyamide yarn with a linear density of 78 grams per 10,000 m (i.e. 78 diex) wherein each ply comprises 68 filaments.

### EXAMPLES

### Example 1(a)

| | | Range wt % |
|---|---|---|
| | | in example |
| INGREDIENT | FUNCTION | formulation |
| Petrolatum (White Petroleum | Emollient | 2-6 |
| Jelly) & paraffin (liquid | | |
| Paraffin) | | |
| Polyglyceryl-6-stearate, | Emulsifier | 5-10 |
| polyglyceryl-6 behenate, | | |
| ceteareth-20 & glyceryl | | |
| stearate | | |
| Glycerin | Humectant | 10-16 |
| Menthol | Cooling agent | 0.5-2.5 |
| Methylsulfonylmethane | Anti-inflammatory | 2-8 |
| Phenoxyethanol | Preservative | 0.1-2 |
| Ethanol | Solubiliser | |
| Citric Acid (20% Solution) | pH modifier | |
| Aqua (Purified Water) | Carrier | 65-70 |
| (to 100%) | | |

### Example 1(b)

| | | Range wt % |
|---|---|---|
| | | in example |
| INGREDIENT | FUNCTION | formulation |
| Petrolatum (White Petroleum | Emollient | 2-6 |
| Jelly) & paraffin (liquid | | |
| Paraffin) | | |
| Polyglyceryl-6-stearate, | Emulsifier | 5-10 |
| polyglyceryl-6 behenate, | | |
| ceteareth-20 & glyceryl | | |
| stearate & acrylate-C₁₀₋₃₀ | | |
| alkyl acrylate crosspolymer | | |
| Glycerin | Humectant | 10-16 |
| Menthol | Cooling agent | 0.5-2.5 |
| Methylsulfonylmethane | Anti-inflammatory | 2-8 |
| Methylparaben & propylparaben | Preservative | 0.1-2 |
| Propylene glycol | Solubiliser | |
| Citric Acid (20% Solution) | pH modifier | |
| Aqua (Purified Water) | Carrier | 65-70 |
| (to 100%) | | |

### Method for Manufacturing the Compositions of the Inventions

The compositions of example 1 were manufactured as follows:
(a) Purified water and glycerin were combined in a reaction vessel with stirring and the resulting mixture (Mixture A) was heated to 70° C.
(b) In a second reaction vessel white petroleum jelly, liquid paraffin, emulsifiers and preservative were combined and the resulting mixture (Mixture B) was heated to 70° C.
(c) Once both mixtures reached 70° C., Mixture B was combined with Mixture A and the resulting mixture (Mixture C) was homogenized for 1 minute at 70° C.
(d) Mixture C was then allowed to cool to room temperature (20 to 25° C.) with stirring.
(e) Menthol crystals were dissolved in ethanol, and subsequently combined with mixture C, to provide Mixture D.
(f) The pH of the Mixture D was then adjusted using citric acid solution to a pH 5.50-6.50.

### Comparative Example 1

| Ingredients | Function | % w/w |
|---|---|---|
| Dipropylheptyl Carboate | Emollient | 10 |
| Sodium Stearoyl Glutamate | Emulsifier (o/w) | 0.2-1.2 |
| Glycerin | Humectant | 4-12 |
| Menthol | Cooling agent | 0.1-5 |
| methylsulfonylmethane | Anti-inflammatory | 2-8 |
| Phenoxyethanol, Ethylhexylglycerine | Preservative | 0.1-2 |
| Sodium Polyacrylate | Thickener | |
| Pentaerythrityl Distearate | | |
| Citric Acid | pH modifier | |
| Aqua | Carrier | 76-80 |

### Comparative Example 2

| Ingredients | Function | % w/w |
|---|---|---|
| Dimethicone, C₁₂₋₁₅ Alkyl Benzoate & | Emollient | 4-6 |
| Cetearyl Alcohol | | |
| Caprylic/Capric Triglyceride, Glyceryl | Emulsifier (o/w) | 4-12 |
| Stearate SE, Steareth-21, Steareth-2 and | | |
| PEG-40 Hydrogenated Castor Oil | | |
| Glycerin | Humectant | 2-8 |
| Menthyl Lactate | Active | 0.1-5 |
| Methylsulfonylmethane | Active | 2-8 |
| Phenoxyethanol, Ethylhexylglycerin & | Preservative | |
| Tetrasodium EDTA | | |
| Tocopheryl Acetate | Antioxidant | |
| Dimethyl Ether | Propellant | 35-45 |
| Water | Carrier | 40-50 |

### Comparative Example 3

| Ingredients | Function | % w/w |
|---|---|---|
| Shea Butter, Cetearyl Alcohol & | Emollient | 10-14 |
| Octyl dodecanol | | |
| C₂₀₋₂₂ alkyl phosphate and C₂₀₋₂₂ alcohols | Emulsifier | 0.2-1.2 |
| Glycerin | humectant | 12-16 |
| Menthol | Cooling agent | 0.1-5 |
| methylsulfonylmethane | Anti-inflammatory | 2-8 |
| Phenoxyethanol and ethylhexyl glycerin | Preservative | |
| Polyacrylamide & C₁₂₋₁₄ Isoparaffin & | Stabilizer | |
| Laureth 7 | | |
| Sodium Hydroxide | pH Adjustment | |
| Water | Carrier | 60-70 |

The compositions of comparative examples 1 to 3 were prepared in a similar manner to those of example 1.

The compositions of Example 1 and Comparative Examples 2 and 3 were assessed for the following properties:
Moisturising and cooling effectiveness
Sprayability
Leakage
Physicochemical stability

The moisturising and cooling effectiveness of the composition was assessed qualitatively. Compositions were applied to the inner layer of twin skin socks after exercise, and participants were asked to score how moisturised their feet felt 1 hour after application of the composition. The scoring scale was from 1 to 10, with 1 being very dry and 10 being very moisturised. Participants were also asked to score how moisturised their feet felt before exercise using the same scoring scale. Participants were also asked to respond to a statement that their skin felt cooled upon application of the compositions by answering strongly agree, agree, neither agree nor disagree, disagree or strongly disagree. Comparative example 2 was deemed ineffective for moisturizing and cooling, whereas example 1 and comparative examples 1 and 3 were effective at moisturising and cooling.

The sprayability of each of the formulations was assessed using an applicator according to WO2019224319, and also using a manual pump action mini trigger spray with pre-compression, specifically a PZ Helios spray pump commercially available from Aptar®. The manual pump was configured to deliver a dose of 0.29 mL per stroke (i.e. per complete depression of the pump head). Another suitable commercially available pump for delivery of the compositions of the invention includes the Raepak TR-12-2-00.

The compositions of example 1 and those of comparative examples 1 and 2 were sprayable using the specified applicator and pre-compression spray pump, however the composition of comparative example 3 was not.

Each of the compositions was assessed for leakage when applied to a twin skin garment i.e. a garment comprising an inner layer and an outer layer.

### Method for Leakage Assessment

1.16 mL of each composition was applied to an area of 2500 mm² measuring 25 mm×100 mm on an inner skin facing surface of the inner layer of the twin skin garment. This treated area was placed in contact with an impermeable surface e.g. 316 polished stainless steel.

A 25 mm wide roller was then rolled along an outer surface of the outer layer of the twin skin garment overlying the treated area along the 100 mm length where the composition was applied. A constant force of 14.6 Newtons was applied to the sock through the roller while rolling. The roller was traversed across the area 20 times in one minute. Thereafter the outer layer of the garment was inspected to assess whether the composition had leaked from the inner layer to the exterior of the outer layer.

### Results

No leakage was observed for the compositions of example 1 and of comparative examples 1 and 3, however, leakage was observed for the composition of comparative example 2.

The next assessment was a physicochemical stability test. Each of the compositions was stored under the following conditions for the specified periods:

| Duration | Temperature | Light exposure |
|---|---|---|
| 2 weeks | 4° C., 40° C., 20° C. | @20° C. |
| 1 month | 4° C., 40° C., 20° C. | @20° C. |
| 2 months | 4° C., 40° C., 20° C. | @20° C. |
| 3 months | 4° C., 40° C., 20° C. | @20° C. |

The compositions were also assessed for stability in a series of five freeze thaw cycles.

The viscosity of the compositions were assessed under the specified conditions, and the compositions were evaluated for changes in colour, pH and fragrance under the specified conditions. Compositions were also visually inspected to assess whether any splitting or layering was observed after testing.

The compositions of example 1 and of comparative example 2 remained within a desired viscosity range under each of the tested conditions, with no changes in colour or fragrance being observed. The compositions of example 1 and of comparative example 2 also remained within a desired pH range. The compositions of example 1 and of comparative example 2 were also re-tested for sprayability after the physicochemical testing was conducted, and both remained sprayable. The compositions of comparative examples 1 and 3 split under some of the test conditions.

TABLE 1
The results for the composition of example 1 are shown below.

| | 2 weeks | | | | 1 month | | | |
|---|---|---|---|---|---|---|---|---|
| Condition | 4° C. | 40° C. | L | C | 4° C. | 40° C. | L | C |
| Colour | 0 | 0 | 0 | 0 | 0 | 0 | 0 | 0 |
| Viscosity | 31090* | 8593* | 35930* | 11250* | 1537** | 1053** | 1200** | 1168** |
| pH | 5.66 | 5.78 | 5.82 | 5.81 | 5.60 | 5.64 | 5.64 | 5.68 |
| Fragrance | 0 | 0 | 0 | 0 | 0 | 0 | 0 | 0 |

| | 2 months | | | | 3 months | | | |
|---|---|---|---|---|---|---|---|---|
| Condition | 4° C. | 40° C. | L | C | 4° C. | 40° C. | L | C |
| Colour | 0 | 0 | 0 | 0 | 0 | 0 | 0 | 0 |
| Viscosity | 1375** | 1247** | 1312** | 1328** | 1352** | 846** | 1506** | 1025** |
| pH | 5.61 | 5.49 | 5.64 | 5.55 | 6.19 | 6.13 | 6.21 | 6.14 |
| Fragrance | 0 | 0 | 0 | 0 | 0 | 0 | 0 | 0 |

L = stored in a window (and therefore exposed to light during hours of daylight) at 20° C.; C = control, stored in dark at 20° C.; Samples at 4° C. were stored in darkness in a refrigerator; Samples at 40° C. were stored in darkness in an incubator.
Fragrance Key: 0 = no change, 1 = very slight change, 2 = slight change, 3 = acceptable change, 4 = considerable change; 5 = extreme change
Colour Key: 0 = no change, D = Darker, P = Paler, Y = yellower, B = bluer
Viscosity:
*measured using a Brookfield Model DV-I + viscometer with an S95 spindle at 3 rpm at 20° C.;
**measured using a Brookfield Model DV-I + viscometer with an S93 spindle at 30 rpm at 20° C.

The compositions of the invention were also evaluated to test the effectiveness of the preservative system within the compositions in accordance with The Polish Norm PN-EN ISO 11930:2019-03 “Cosmetics—Microbiology—Test of effectiveness assessment of preservation of cosmetic products”.

The composition of example 1 was tested as outlined below:

### 1. THE AIM/SCOPE OF THE TEST

Confirmation of selecting the proper concentration of the preservative in the laboratory test, (so called challenge test).

The process of cosmetics preservation aims at maintaining microbiological purity, protecting from secondary infection during application and storage and preventing the development of microorganisms in the preparations intended for repeated use.

### 2. THE METHOD OF CONDUCTING THE TEST

The test of effectiveness of the applied preservative was conducted on the basis of:
Polish Norm PN-EN ISO 11930:2019-03 “Cosmetics—Microbiology—Test of effectiveness assessment of preservation of cosmetic products”

The tests were carried out to verify, whether the preparation shows the appropriate preserving properties.

The preserving properties of the preparation are considered as appropriate if under the test conditions, after a defined time and in the defined temperature in the inoculated preparation occurs a significant decrease of living microorganism cells or the growth of the cells does not occur at all, as required. The criteria of acceptance within the scope of reducing the number of microorganism cells in time are different for various categories of preparations. According to the information provided in the standard, the A and B criteria are applied. The A criterion refers to the products, with reference to which an infection of the product is probable. The B criterion is applied when the degree of the product's protection is acceptable and there's little probability of infecting the product.

According to the requirements, the criteria of recommended anti-microorganism effectiveness are expressed as logarithm of the reduction in number of microorganisms (Table (i) below):

reduction Log (Rₓ = IgN₀ - IgNₓ) required^{a}

| | Microorganisms | | | | | | | |
|---|---|---|---|---|---|---|---|---|
| | Bacteria | | | Candida albicans | | | Aspergillus niger | |
| | Time of sample | | | | | | | |
| storing | T 7 | T 14 | T 28 | T 7 | T 14 | T 28 | T 14 | T 28 |
| A criterion | 23 | ≥3 i NI^{b} | ≥3 i NI | ≥1 | ≥1 i NI | ≥1 i NI | ≥0^{c} | ≥1 |
| B criterion | not | ≥3 | ≥3 i NI | not | ≥1 | ≥1 i NI | ≥0 | ≥0 i NI |
| | performed | | | performed | | | | |

^{a}the acceptable range of deviation is 0.5 log
^{b}NI— no growth in number of microorganisms compared to the previous period of contact
^{c}Rₓ =) when IgN₀ = IgNₓ (no growth since initial counting)

### 2.1 Methodology of the Test'S Performance

### a) The Method'S Principle on the Basis of: PN-EN ISO 11930:2019-03

Determining the number of living microorganism cells in the tested sample of the product vaccinated with the inoculum of the appropriate test microorganisms, planted from this sample at strictly defined intervals.

The tests were performed on the basis of the PN-EN ISO 11930:2019-03.

### b) Test Organisms

Escherichia coli (A TCC 8739)
Staphylococcus aureus (A TCC 9027)
Pseudomonas aeruginosa (ATCC 6538)
Candida albicans (A TCC 10231)
Aspergillus brasiliensis (ATCC 16404)

### c) Culture Media

Tryptone Soya Agar (TSA)
Sabouraud Agar
Potato Dextrose Agar (PDA)
DIE Neut Broth

### d) The Contents of the Neutralizer DIE Neut Broth

pancreatin digest of casein
Yeast extract
dextrose
sodium thioglycollate
sodium tiosulfate
lecithin
Tween 80
Bromocresol purple

### e) The Conditions of Storing the Samples

The samples of the product with the addition of test strains were stored during the test in a dark place, at the temperature 18-27° C.

### f) Time and Temperature of Incubation

The inoculations from the tested samples were incubated:
Candida albicans bacteria temperature 30-35° C. for 48-72 h
Aspergillus brasiliensis temperature 20-25° C. for 3-5 days

### g) Counting of Colonies

After incubation the colonies on the incubated dishes were enumerated. For all enumerations the dishes containing between 30 and 300 colonies for bacteria and Candida albicans and between 15 and 150 colonies for Aspergillus brasiliensis were taken.

### h) Preparation of the Inoculums of the Infection Test Strains

From the culture on agar substrata the strains were carried with an inoculating loop to separate ampoules containing 2 ml of sterile physiological solution each. Microorganism suspensions (bacteria and Candida albicans) of densities specified on the McFarland scale were prepared with the use of a densimat. Inoculum of the Aspergillus brasiliensis strain was prepared in accordance with the instruction supplied by the BioMerieux company. After removing the cap from the rehydration fluid and the cover from the glass ampoule containing BioBall, the BioBall was carried into the rehydration fluid, covered with the cap and left for 30 seconds. Next, it was mixed with vortex type of stirrer for 5 seconds.

In order to obtain the infecting suspensions with densities specified in the PN-EN ISO 11930:2019-03 norm (1×10⁷-1×10⁸ cfu/ml for bacteria and 1×10⁶-1×10⁷ cfu/ml for fungi) a decimal dilution (10-1) was prepared for bacteria and the Aspergillus brasiliensis strain. In the case of Candida albicans, the infecting suspension was the non-diluted suspension of a density set with the use of the McFarland scale.

### 2.2. The Course of the Test

The test was carried out in two stages. The first of them aimed at verifying the microbiological purity of the tested product, the second stage was the essential test (challenge test).

### Stage I (Preliminary)

Before entering the essential test (stage II) the tested product was verified from the point of view of microbiological purity. This test aimed at affirming the possible microbiological impurities of the product arising, among others, from production stages, taking samples, storage and transport. The results of tests were compiled in table (ii) below.

TABLE II
The results of the tests on microbiological
purity of the tested product

| | | Result of | |
|---|---|---|---|
| No. | Type of test | determination | Analytical method |
| 1. | Total aerobic | <10 cfu/g | PN-EN ISO 21149: 2017 |
| | mesophilic | | July excluding the |
| | bacteria A | | p.9.3.2.2, 9.3.2.3 and 9.4 |
| 2. | Total yeast and | <10 cfu/g | PN-EN ISO 16212: 2017 |
| | moulds A | | August excluding the |
| | | | p.9.3.2.2, 9.3.2.3 |
| 3. | Presence of | Absent | PN-EN ISO 22717: 2016 |
| | Pseudomonas | | |
| | aeruginosa A | | |
| 4. | Presence of | Absent | PN-EN ISO 22718: 2016 |
| | Staphylococcus | | |
| | aureus A | | |
| 5. | Presence of | Absent | PN-EN ISO 21150: 2016 |
| | Escherichia coli A | | |
| 6. | Presence of | Absent | PN-EN ISO 18416: 2016 |
| | Candida albicans A | | |

The performed tests revealed, no present microbiological contamination with aerobic mesophiles (bacteria), and no presence of such pathogenic microorganisms such as: Staphylococcus aureus, Pseudomonas aeruginosa and Candida albicans was detected. In connection with the above, the II stage of the test was undertaken—the challenge test.

### Stage II (the Essential Test)

The main purpose of the tests was to confirm the effectiveness of a preservative with reference to the test microorganisms.

The applied test strains of the Aspergillus brasiliensis (niger) strain was supplied in the form of a BioBall by the BioMerieux company.

### 3. EXECUTION OF THE TEST

### Determination of the Neutralizer'S Effectiveness

The determination of the neutralizer's effectiveness was carried out in order to verify, whether the applied neutralizer has the capacity to neutralize the preservative without inhibiting the growth of microorganisms. With this end in view, the number of microorganisms defined in the norm was incubated in the presence and in the absence of the product subjected to the test and the neutralizer. The applied neutralizer is considered as effective, if the recovery of microorganisms in the presence of the product amounts to at least 50%, with relation to the number of microorganisms not subjected to the effect of the tested product. The results of determination of the neutralizer's effectiveness were given in the Table (iii).

TABLE III
Determination of the neutralizer's effectiveness

| | | Control sample | | |
|---|---|---|---|---|
| | | (neutralizer | Sample with | |
| | | without the | the addition | |
| | The amount | addition of the | of the tested | |
| | of strain | product) Nᵥₙ | product N_{vf} | |
| | (units/ml) Nv | (dilution 10¹⁰) | (dilution 10⁰) | |
| Test strain | (dilution 10⁰) | (cfu/ml) | (cfuml) | 0.5 × Nᵥₙ |
| Staphylococcus | 109 | 107 | 101 | 51 |
| aureus | | | | |
| (ATCC 6538) | | | | |
| Pseudomonas | 99 | 96 | 91 | 46 |
| aeruginosa | | | | |
| (ATCC 9027) | | | | |
| Escherichia coli | 105 | 101 | 96 | 48 |
| (ATCC 8739) | | | | |
| Candida albicans | 103 | 99 | 96 | 48 |
| ATCC 10231) | | | | |
| Aspergillus | 97 | 95 | 89 | 45 |
| brasiliensis | | | | |
| (ATCC16404) | | | | |

In the case of all verified strains, the neutralizer's effectiveness should amount to over 50% i.e., meet the N_{vf}>0.5 Nᵥₙ condition, and the number of microorganisms from the control sample (Nᵥₙ) has to be close to the number in the Nᵥ strain.

Validation conditions concerning the effect of the neutralizer were fulfilled.

Preliminary determination of the number of cells in the infecting suspension of the test strain. In order to define the precise number of cells in the infecting suspensions (N) used in the further part of the test, decimal dilutions were prepared from the suspensions, and then pour plate cultures were carried out from the selected dilutions (10⁻⁵-10⁻⁶). The plates were incubated for 24-48 h in the temp. +30-35° C. (for bacteria and Candida albicans) and for 3-5 days in the temp. +20-25° C. (for Aspergillus niger).

TABLE IV
Density of the infecting suspension (N)
and the number of cells at time 0 (N₀).

| | The number of | |
|---|---|---|
| | microorganisms | The initial |
| | in the infection | number of cells |
| Inoculum for | inoculum in cfu/ml | (N₀) derived from |
| the test strain | (N) | the N/10⁰ formula |
| Staphylococcus aureus | 9.8 × 10⁷ | 9.8 × 10⁵ |
| (ATCC 6538) | | |
| Pseudomonas aeruginosa | 6.7 × 10⁷ | 6.7 × 10⁵ |
| (ATCC 9027) | | |
| Escherichia coli | 1.1 × 10⁸ | 1.1 × 10⁶ |
| (ATCC 8739) | | |
| Candida albicans | 1.0 × 10⁷ | 1.0 × 10⁵ |
| ATCC 10231) | | |
| Aspergillus brasiliensis | 7.1 × 10⁶ | 7.1 × 10⁴ |
| (ATCC16404) | | |

### Determining Effectiveness of the Preservative

The test consisted in the vaccination of the tested sample with the inoculum of the test strains.

20 g of the test sample was weighed in separate containers and then 0.2 ml of inoculum of the test strains was added to each separate container. Next, the containers were shaken up, thereby distributing the suspension of microorganisms. After inoculation the number of microorganisms (N₀) recommended by the standard in 1 g of the product is as specified below—for results see Table (iv):
for bacteria 1×10⁵ to 1×10⁶
for fungi 1×10⁴ to 1×10⁵

### Determining the Degree of Reduction of Test Microorganisms After the Specified Period of Contact With the Cosmetic Containing the Preservative

In the test was defined the number of test microorganism cells after the specified periods of the preparation's contact with the inoculated test microorganisms. The periods of contact were, respectively: 7 days, 14 days and 28 days for the bacteria and Candida albicans and 14 and 28 days for Aspergillus niger. In order to define the number of living cells in 1 g of the given product, the inoculations of the product by pour plate method were performed, with the application of the thinner defined by the PN-EN ISO 11930:2019-03 standard. Before inoculation on Petri plates, the product was left in the thinner for 30±15 minutes at the room temperature. The test results were compared in the tables 5 and 6.

TABLE V
The results of microorganism growth
after the specified period of contact

| | Inoculation after | Inoculation after | Inoculation after |
|---|---|---|---|
| Test strains | 7 days (cfu/g) | 14 days (cfu/g) | 28 days (cfu/g) |
| St. aureus | <10 | <10 | <10 |
| (ATCC 6538) | | | |
| Ps. Aeruginosa | <10 | <10 | <10 |
| (ATCC 9027) | | | |
| E. coli | <10 | <10 | <10 |
| (ATCC 8739) | | | |
| C. albicans | <10 | <10 | <10 |
| (ATCC 10231) | | | |
| Aspergillus | — | <10 | <10 |
| brasilensis | | | |
| (ATCC 16404) | | | |

TABLE VI
Logarithm of reduction in number of microorganisms after
the specified period of contact calculated from the formula: Rₓ =
IgN₀ − IgNₓ (where: Rx - reduction value, IgN₀ - number of
microorganisms in time t₀, IgNx - number of microorganisms
after the specified period of contact)

| | Logarithm of reduction in number of | | |
|---|---|---|---|
| | microorganisms after a period of contact (Rₓ): | | |
| Test strains | 7 days | 14 days | 28 days |
| St. aureus | >5 | >5 | >5 |
| (ATCC 6538) | | | |
| Ps. Aeruginosa | >5 | >5 | >5 |
| (ATCC 9027) | | | |
| E. coli | >5 | >5 | >5 |
| (ATCC 8739) | | | |
| C. albicans | >4 | >4 | >4 |
| (ATCC 10231) | | | |
| Aspergillus | — | >4 | >4 |
| brasilensis | | | |
| (ATCC 16404) | | | |

### 4. CONCLUSIONS

On the basis of the obtained test results for the test compositions we can affirm, that in the case of test strains: Staphylococcus aureus (A TCC 6538), Escherichia coli (A TCC 8739), Pseudomonas aeruginosa (A TCC 9027), Candida albicans (A TCC 10231) and Aspergillus brasiliensis (ATCC16404) the requirements for the preparations laid down in the PN-EN ISO 11930:2019-03 (A criterion and B criterion) confirming the appropriate preserving properties of the cosmetic towards these pathogens have been fulfilled.

The efficacy of a composition as described in example 1 was trialled in confidence in 21 endurance athlete subjects. 10 mL of the composition was sprayed onto an inner surface of the inner layer (skin contacting surface) of twin skin socks, and the socks were worn for 1 hour after exercise.

Prior to exercising subjects were asked to score from 1 to 10 how moisturised their feet were before the composition of the invention was applied, with a scope of 1 being very dry and a score of 10 being very moisturised.

1 hour after wearing the socks to which the composition of the invention had been applied, subjects were asked to again score how moisturised their feet were.

| Score | 1 | 2 | 3 | 4 | 5 | 6 | 7 | 8 | 9 | 10 |
|---|---|---|---|---|---|---|---|---|---|---|
| Before | 1 | 2 | 4 | 5 | 4 | 2 | 3 | 0 | 0 | 0 |
| exercise | | | | | | | | | | |
| 1 hour | 0 | 0 | 0 | 0 | 0 | 1 | 5 | 5 | 8 | 2 |
| After | | | | | | | | | | |
| exercise | | | | | | | | | | |

Scoring level: 1 = very dry; 10 = very moisturised

A significant improvement was experienced across the group.

The same group of subjects were also asked to score the level of aches/pains felt in feet directly after exercise and before application of the composition, with a score of 1 being pain free and a score of 10 being very painful. 1 hour after application of the composition, subjects were asked to score the level of aches/pains felt in the feet.

| Score | 1 | 2 | 3 | 4 | 5 | 6 | 7 | 8 | 9 | 10 |
|---|---|---|---|---|---|---|---|---|---|---|
| Before | 0 | 2 | 3 | 4 | 5 | 2 | 3 | 1 | 0 | 1 |
| application | | | | | | | | | | |
| 1 hour | 6 | 6 | 4 | 2 | 0 | 0 | 1 | 1 | 1 | 0 |
| after | | | | | | | | | | |
| application | | | | | | | | | | |

A marked improvement in the level of aches/pains felt in the feet was observed across the group of subjects.

The following table exemplifies the efficacy of the composition of the invention

| | Strongly | | Neither agree | | Strongly |
|---|---|---|---|---|---|
| | Agree | Agree | nor disagree | disagree | disagree |
| I felt less aches and | 5 | 15 | 1 | 0 | 0 |
| pains after application of | | | | | |
| the trial composition | | | | | |
| My skin felt cooled upon | 17 | 3 | 1 | 0 | 0 |
| application of the trial | | | | | |
| composition | | | | | |
| My skin felt moisturised | 10 | 11 | | 0 | 0 |
| and hydrated after | | | | | |
| application of the trial | | | | | |
| composition | | | | | |
| My recovery was improved | 8 | 12 | 1 | 0 | 0 |
| My recovery time was | 8 | 10 | 3 | 0 | 0 |
| reduced | | | | | |
| Targeted relief was felt | 8 | 13 | 0 | 0 | 0 |
| where the trial | | | | | |
| composition was applied | | | | | |

The moisturising capability of a composition of the invention, such as that described in Example 1 was assessed in a confidential trial involving a panel of 10 subjects over the course of 48 hours.

Skin moisture is measured using electric skin properties. The methods are based on the conductance, impedance and capacitance of the stratum corneum. Capacitance is the established standard method (e.g. corneometry) for measuring skin moisture. The principle of measurement is based on the dielectric constant of water, which strongly differs from that of other substances. The capacitor's electric field, influenced by the scatter field in the moist stratum corneum, is altered proportionally to the moisture content. The higher the measured capacitance value the higher the moisture content.

The moisture level of the skin of each subject was measured using a Corneometer® CM 825, available from Courage+Khazaka Electronic GmbH. The Corneometer® CM 825 measures the change in the dielectric constant due to skin surface hydration by capacitance differences of a precision capacitor. The measurement depth is very small (10-20 μm of the Stratum corneum) to exclude the influence of water in deeper skin layers.

Each of the 10 subjects was symmetrically marked on the inside of both forearms (at a distance of about 5 cm from the wrist flexion) with 3 squares of 3×3 cm each. The squares on one of the forearms were control squares. The squares on the other arm were the test squares to which the tested cosmetic was applied evenly in an amount of 0.003 g/cm².

Immediately before applying the cosmetic, skin hydration was measured in each square. Subsequent measurements of skin hydration were made at the following intervals after application of the composition of the invention: 1, 3, 7, 24 and 48 hours. Moisture levels were measured in triplicate, and an arithmetic average taken as the final result for each assessment. The assessment was carried out in an air-conditioned room with a maintained temperature of 20±2° C. and a relative humidity of 50±10%.

The average results of measurements of skin hydration for individual subjects measured corneometrically over a 48 hour period from a single application of the composition of the invention are summarised in the below table and as shown in FIG. 1.

| | Forearm skin hydration in m.u. [Corneometer ® CM 825] | | | | | |
|---|---|---|---|---|---|---|
| | | After 1 h | After 3 h | After 7 h | After 24 h | After 48 h |
| Subject | P₀ | P_{K} | P_{K} | P_{K} | P_{K} | P_{K} |
| 1 | 31 | 65 | 60 | 57 | 37 | 32 |
| 2 | 34 | 63 | 59 | 57 | 44 | 35 |
| 3 | 36 | 65 | 60 | 53 | 42 | 37 |
| 4 | 35 | 65 | 60 | 54 | 41 | 35 |
| 5 | 30 | 65 | 59 | 54 | 37 | 31 |
| 6 | 42 | 69 | 62 | 54 | 46 | 43 |
| 7 | 38 | 66 | 64 | 55 | 43 | 38 |
| 8 | 29 | 68 | 63 | 57 | 36 | 34 |
| 9 | 38 | 73 | 64 | 57 | 43 | 39 |
| 10 | 36 | 63 | 61 | 56 | 43 | 37 |
| Mean value | 35 | 66 | 61 | 55 | 41 | 36 |
| % increase | | 89 | 74 | 49 | 17 | No |
| in moisture | | | | | | change |
| levels | | | | | | |

P₀ = measurement of moisture level on skin not treated with the test product, performed immediately before application
Pₖ = measurement of moisture level on skin treated with tested product, performed at the specified time after application
* average of values measured in triplicate before applying the tested product
m.u. = measured units (i.e. arbitrary units)

Advantageously, within 1 hour of application to the skin, skin surface hydration had increased by over 80% in comparison to the level of skin surface hydration prior to application of the composition of the invention. Within 3 hours of application to the skin, skin surface hydration remained over 70% higher than the level of skin surface hydration prior to application of the composition of the invention. Advantageously, after 7 hours of application to the skin, skin surface hydration remained over 45% higher than the level of skin surface hydration prior to application of the composition of the invention. After 24 hours of application to the skin, skin surface hydration remained over 15% higher than the level of skin surface hydration prior to application of the composition of the invention.

The optimal composition was assessed in conjunction with a variety of garments, in particular with twin skin garments i.e. garments comprising an inner layer and an outer layer. The inner layer and outer layer are separate layers. The inner layer and outer layer may be connected, for example, they may be knitted together, or adhered or otherwise joined.

The most effective garment comprised an inner layer formed from knitted yarn, wherein the yarn comprised 2-ply multifilament nylon 66 yarn having a linear density of 78 dtex, wherein a single ply of said yarn comprised 68 filaments i.e. 2/78f68 nylon 66. The inner layer and the knitted yarn also comprised 2-ply multifilament elastane having a linear density of 78 dtex, wherein a single ply of said yarn comprised 23 filaments i.e. 2/78f23 elastane.

An inner layers of 2/78f68 nylon 66 on its own was somewhat less effective than an inner layer composed of both nylon 66 and elastane. And both nylon 66 on its own and nylon 66 with elastane proved far superior to an inner layer composed of polypropylene.

Optionally, the outer layer may comprise polypropylene. For example, the outer layer may comprise 2-ply multifilament polypropylene yarn, having a linear density of 120 dtex, and wherein a single ply of said yarn comprised 33 filaments i.e. 2/120f33 polypropylene.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

1. An oil-in-water emulsion composition comprising:
(ii) an emollient which is present in an amount of from 1 wt % to 9 wt % based on the total weight of the composition;
(iii) a humectant comprising glycerine which is present in an amount of from 2% to 20 wt % based on the total weight of the composition;
(iv) a cooling agent selected from the group consisting of menthol, menthyl lactate, menthyl acetate, levomenthol, one or more menthol esters, and methyldiisopropyl propionamide; wherein said cooling agent is present in an amount of from 0.1 wt % to 5 wt %, optionally 0.1 wt % to 3 wt % based on the total weight of the composition;
(v) an anti-inflammatory agent comprising dimethyl sulfone which is present in an amount of from 0.2 wt % to 10 wt % based on the total weight of the composition;
(vi) an emulsifier which is present in an amount of from 5 wt % to 10 wt %;
(vii) and water which is present in an amount of from 60 wt % to 75 wt % based on the total weight of the composition.

2. The oil-in-water emulsion composition according to claim 1, wherein the emollient is present in an amount of from 1 wt % to 5 wt % based on the total weight of the composition, suitably from 1.5 wt % to 4.5 wt %, preferably from 1.5 wt % to 3.5 wt % based on the total weight of the composition.

3. The oil-in-water emulsion composition according to claim 1 or 2, wherein the emollient comprises at least one of: mineral oil, petrolatum, paraffin, ozokerite, microcrystalline wax, polyethylene, squalene and perhydrosqualene.

4. The oil-in-water emulsion composition according to claim 3, wherein the emollient comprises two or more of: mineral oil, petrolatum, and paraffin, preferably wherein the emollient is petrolatum and paraffin.

5. The oil-in-water emulsion composition according to any preceding claim, wherein the emulsifier is present in an amount of from 5 wt % to 10 wt %.

6. The oil-in-water emulsion composition according to any preceding claim, wherein the emulsifier is selected from one or more of:
(i) polyethylene glycol ethers of the general formula:
R-O—(—CH₂—CH₂—O—)n-R′, where R and R′, independently of one another, are branched or unbranched alkyl or alkenyl radicals and n is a number from 10 to 80,
(ii) polyglyceryl fatty acid esters, and
(iii) optionally an acrylate-C₁₀₋₃₀ acrylate crosspolymer.

7. The oil-in-water emulsion composition according to claim 6, wherein the emulsifier comprises a polyethylene glycol ether of alcohols selected from isocetyl alcohol (e.g., Isoceteth-20), isostearyl alcohol (e.g., Isosteareth-20), cetyl alcohol (e.g., Ceteth-20), oleyl alcohol (e.g., Oleth-20) and cetearyl alcohol (e.g., Ceteareth-20).

8. The oil-in-water emulsion composition according to claim 6 or 7, wherein the emulsifier comprises at least one polyethylene glycol ether and at least one polyglyceryl fatty acid ester, for example, wherein the emulsifier comprises glyceryl stearate, and polyglyceryl-6-stearate, or wherein the emulsifier comprises glyceryl stearate, ceteareth-20, polyglyceryl-6-stearate, and polyglyceryl-6 behenate.

9. The oil-in-water emulsion composition according to any preceding claims, further comprising a preservative.

10. The oil-in-water emulsion composition according to claim 5, wherein the preservative is present in an amount of from about 0.01 wt % to 5 wt %, preferably from 0.1% to 2 wt %, such as from 0.5 wt % to 1.5 wt % based on the total weight of the composition.

11. The oil-in-water emulsion composition according to claim 5 or 6, wherein the preservative comprises at least one of: a parahydroxybenzoic acid, methylparaben, propylparaben, benzyl alcohol, phenoxyethanol, ethylhexylglycerin, octanediol, hexanediol, pentandiol, sorbitan caprylate, capralyl glycol, caprylhydroxamic acid, phenoxyethanol triethylene glycol, sodium benzoate, and 2-bromo-2-nitro-1,3-propanediol (bronopol).

12. The oil-in-water emulsion composition according to any preceding claim, further comprising a pH modifier.

13. The oil-in-water emulsion composition according to any preceding claim wherein the glycerine is present in an amount of from 10 wt % to 16 wt % based on the total weight of the composition, preferably from 12 wt % to 14 wt % based on the total weight of the composition.

14. The oil-in-water emulsion composition according to any preceding claim, wherein the cooling agent is present in an amount of from 0.5 to 1.5 wt % based on the total weight of the composition, preferably from 0.8 to 1.2 wt % based on the total weight of the composition.

15. The oil-in-water emulsion composition according to any preceding claim, wherein the dimethyl sulfone is present in an amount of from 2 to 8 wt % based on the total weight of the composition, preferably from 4 to 7 wt % based on the total weight of the composition.

16. The oil-in-water emulsion composition according to any preceding claim, wherein the water is present in an amount of from 63 to 73 wt % based on the total weight of the composition, preferably from 65 to 70 wt % based on the total weight of the composition.

17. The oil-in-water emulsion composition according to any preceding claim, wherein the composition has a pH in the range of from 5.0 to 7.0, preferably 5.3 to 6.3.

18. The oil-in-water emulsion composition according to any preceding claim, wherein the composition has a viscosity in the range of from 500 mPa·s to 35000 mPa·s when measured at 20° C. using a Brookfield viscometer with a type S93 spindle and a rotation speed of 30 rpm, preferably wherein the viscosity is in the range of from 500 mPa·s to 3,000 mPa·s when measured at 20° C. using a Brookfield viscometer with a type s93 spindle and a rotation speed of 30 rpm.

19. The oil-in-water emulsion composition according to any preceding claim, wherein said composition is stable with respect to microbiological burden, for at least 6 months when stored in a sealed container at accelerated storage conditions (40° C. and 75% Relative Humidity).

20. A method for moisturising skin of a subject, comprising the following steps:
(i) spraying an oil-in-water composition according to any one of claims 1 to 20 onto a garment, and
(ii) applying said garment such as a sock or sleeve to the skin of said subject, such that the oil-in-water composition applied to said garment wets the skin of said subject.

21. The method of claim 20, wherein the garment comprises an inner layer and an outer layer.

22. The method of claim 21, wherein the inner layer comprises a polyamide material, preferably nylon 66 (poly[imino(1,6-dioxohexamethylene) iminohexamethylene]).

23. The method of claim 22, wherein the inner layer further comprises up to 10 wt % elastane based on the total weight of the inner layer, suitably from 0.5 wt % to 10 wt % elastane based on the total weight of the inner layer, for example from 1 wt % to 5 wt %.

24. The method of claims 21 to 23, wherein the inner layer is formed from knitted yarn.

25. The method of claim 24, wherein the knitted yarn includes 2-ply yarn with a linear density in the range of from 50 dtex to 100 dtex.

26. The method of claim 24 or 25, wherein the knitted yarn includes 2-ply yarn wherein a single ply of said yarn comprises from 50 to 90 filaments, preferably from 60 to 80 filaments, such as from 65 to 75 filaments.

27. The method of any one of claims 22 to 26, wherein the inner layer comprises nylon 66 and elastane, for example, wherein the inner layer comprises nylon 66 in an amount of from 80 wt % to 99 wt %, such as from 90 to 99 wt % based on the total weight of the inner layer, and elastane in an amount of from 0.5 wt % to 10 wt %, such as from 1 wt % to 5 wt % based on the total weight of the inner layer.

28. The method of claim 27, wherein the inner layer is formed from knitted yarn, wherein the yarn comprises 2-ply nylon 66 yarn and 2-ply elastane yarn.

29. The method of claim 28, wherein the 2-ply nylon 66 yarn has a linear density in the range of from 50 dtex to 100 dtex, preferably from 75 dtex to 85 dtex, such as from 75 to 80 dtex; and/or wherein the 2-ply elastane yarn has a linear density in the range of from 50 dtex to 100 dtex, preferably from 75 dtex to 85 dtex, such as from 75 to 80 dtex.

30. The method of any one of claim 28 or 29, wherein a single ply of the 2-ply nylon 66 yarn comprises from 50 to 90 filaments, preferably from 60 to 80 filaments, such as from 65 to 75 filaments, for example 68 filaments; and/or wherein a single ply of the 2-ply elastane yarn comprises from 15 to 35 filaments, preferably from 20 to 30 filaments, such as from 20 to 25 filaments, for example 23 filaments.

31. A garment such as a sleeve, glove or sock comprising an inner layer and an outer layer, wherein a composition according to any one of claims 1 to 19 is partially coated on an inner surface of said inner layer, said inner surface being a skin facing surface when said garment is worn by a user.
